(19) United States
(12) Patent Application Publication
(10) Pub. No.: US 2014/0065679 A1
(43) Pub. Date: Mar. 6, 2014

(54) PRODUCTION OF ENANTIOMERICALLY PURIFIED AMINO ACIDS

(71) Applicants: Scott Baxter, Edinburgh, Scotland (GB); Dominic Campopiano, Edinburgh, Scotland (GB); Karen Elizabeth Holt-Tiffin, Royston, Hertfordshire (GB)

(72) Inventors: Scott Baxter, Edinburgh, Scotland (GB); Dominic Campopiano, Edinburgh, Scotland (GB); Karen Elizabeth Holt-Tiffin, Royston, Hertfordshire (GB)

(73) Assignees: THE UNIVERSITY OF EDINBURGH, Edinburgh, Scotland (GB); DR. REDDY'S LABORATORIES (EU) LIMITED, Beverly, East Yorkshire (GB)

(21) Appl. No.: 14/009,758

(22) PCT Filed: Apr. 10, 2012
(86) PCT No.: PCT/IB12/00836

§ 371 Date: Nov. 20, 2013

Related U.S. Application Data
(60) Provisional application No. 61/474,455, filed on Apr. 12, 2011.
(60) Provisional application No. 61/576,381, filed on Dec. 16, 2011.

(51) Int. Cl. C12P 13/04; C12P 13/22; C12P 13/06

(52) U.S. Cl. CPC C12P 13/04; C12P 13/06; C12P 13/222
USPC 435/108; 435/233; 435/106; 435/116; 435/115

(57) ABSTRACT

The present application relates to a mutated Amycoiatopsis sp. TS-1-60 NAAAR that shows improved activity of the enzyme compared with the wild type Amycoiatopsis sp. TS-1-60 NAAAR. The mutated NAAAR is almost five times more active than its wild type counterpart. The present application also relates to the use of mutated Amycoiatopsis sp. TS-1-60 NAAAR in the production of enantiomerically pure amino acid from its N-acyl derivative via dynamic kinetic resolution method.

### FIELD

The present application relates to the production of enantiomerically purified α-amino acids, via dynamic kinetic resolution of racemic N-acyl amino acids as starting materials, involving the use of an amino acylase enzyme.

### BACKGROUND

Enantiomerically pure D- and L-amino acids are important building blocks in synthetic organic chemistry. They are also important for parenteral nutrition. Many methods of producing enantiomerically purified amino acids are known in the literature. Among them, enzymatic preparation of amino acids is common, as it produces amino acids having high optical purity.

One of the methods of producing amino acids with high optical purity involves deacylation of racemic N-acyl amino acids, using an amino acylase enzyme. As the enzyme preferably acts only on a specific isomer and does not act on the other isomer, the reaction gives a high enantiomeric purity. This process of production of enantiomerically pure compounds is known as kinetic resolution. But a main disadvantage of this reaction is that only 50% yield is possible, unless the unwanted enantiomer is separated from the reaction mixture and reused.

Dynamic kinetic resolution methods of producing an enantiomerically pure compound are defined as processes in which the unwanted isomer can racemize under the reaction conditions. Hence, the reaction can proceed up to about 100% yield, provided the racemization is much faster compared to the rate of the irreversible reaction. Hence, dynamic kinetic resolution method for the synthesis of enantiomerically pure amino acids are chosen, over kinetic resolution methods.

In dynamic kinetic resolution methods, the reaction between a racemic N-acyl amino acid and an acylase enzyme uses N-acyl amino acid racemase (NAAAR) biocatalysts to increase the yield of the enantiomerically pure amino acid significantly. As a result, the process becomes industrially viable at a commercial scale. The concept of using NAAAR can be schematically represented by Schemes 1 and 2, where Scheme 1 represents an acylase based resolution of an L-amino acid from N-acyl-DL-amino acid and Scheme 2 represents an acylase and NAAAR coupled dynamic kinetic resolution.

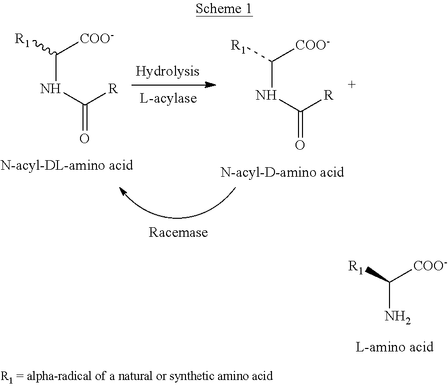

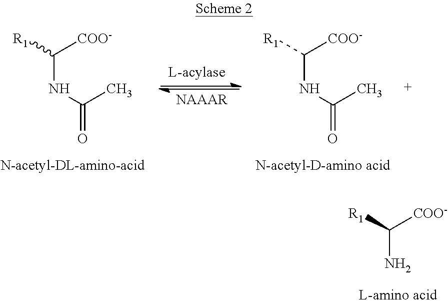

In conventional kinetic resolution methods, L-acylase acts on racemic DL-amino acid and produces N-acyl-D-amino acid and L-amino acid. When NAAAR is added to the reaction mixtures, N-acyl-D-amino acid produced by the forward reaction is racemized to N-acyl-DL-amino acid, and thus the reaction continues until it is about 100% complete.

U.S. Pat. No. 6,656,710 B2 relates to processes for preparing enantiomerically pure amino acids from N-protected amino acids, by the use of an acylase/racemase system. The NAAAR used for the reaction is selected from a group of consisting of Streptomyces atratus Y-53 NAAAR, Amycolatopsis sp. TS-1-60 NAAAR, and Amycolatopsis orientalis sub-species lurida NAAAR.

U.S. Pat. No. 5,525,501 A relates to a DNA fragment containing a gene encoding NAAAR, a vector with the DNA fragment inserted therein, and a microorganism transformed with the vector and capable of producing NAAAR.

U.S. Pat. No. 6,664,803 B2 relates to a method for racemizing N-acylamino acids using an NAAAR, and further to a method for reacting the racemized N-acylamino acids with acylase enzyme to produce enantiomerically pure amino acids. The NAAAR used for racemization has been derived from Sebekia benihana.

U.S. Pat. No. 6,372,459 B1 relates to NAAAR isolated from Amycolatopsis orientalis sub-species lurida. The patent also relates to a method for producing enantiomerically pure amino acids from racemic N-acetyl amino acid by using NAAAR isolated from Amycolatopsis orientalis sub-species lurida.

Tokuyama et al., Appl. Microbiol. Biotechnol. 1994, 40, 853, discloses the purification and properties of NAAAR isolated from Amycolatopsis sp. TS-1-60.

Most of the NAAARs known in the literature are of the wild type. The activity of these wild type NAAARs are very low comparable to the activity of acylase enzyme. As explained earlier, the rate of racemization reaction by NAAARs should be much faster than that of the acylase enzyme to make the method of production of enantiomerically pure amino acids from their N-acyl racemic amino acid derivatives commercially feasible.

### SUMMARY

An aspect of the present application relates to a mutated Amycolatopsis sp. TS-1-60 NAAAR that shows improved activity compared with the wild type Amycolatopsis sp. TS-1-60 NAAAR.

An aspect of the present application relates to a mutated Amycolatopsis sp. TS-1-60 NAAAR that has a wide range of substrate specificity.

An aspect of the present application relates to a mutated Amycolatopsis TS-1-60 NAAAR showing no substrate inhibition up to about 300 mM substrate, so that the mutated Amycolatopsis sp. TS-1-60 NAAAR can be used at higher concentration levels.

An aspect of the present application relates to the use of mutated Amycolatopsis sp. TS-1-60 NAAAR for the racemization of N-acyl amino acid at a commercial scale.

An aspect of the present application relates to the use of mutated Amycolatopsis sp. TS-1-60 NAAAR for producing enantiomerically pure amino acids from a reaction of N-acyl amino acid with an acylase enzyme.

An aspect of the present application relates to processes for producing enantiomerically pure amino acids via a dynamic kinetic resolution process, comprising reacting N-acyl-DL-amino acid with acylase in the presence of mutated Amycolatopsis sp. TS-1-60 NAAAR.

### DETAILED DESCRIPTION

An aspect of the present application provides a mutated Amycolatopsis sp. TS-1-60 NAAAR which shows improved activity of the enzyme compared with the wild type Amycolatopsis sp. TS-1-60 NAAAR. The wild type Amycolatopsis sp. TS-1-60 NAAAR has been mutated at two positions namely, G291D and F323Y. Surprisingly, it is found that the enzymatic activity has been increased by approximately five times that of the activity of the wild type. The sequence of the mutated Amycolatopsis sp. TS-1-60 NAAAR (NAAAR G291D F323Y) is as follows as SEQ ID No. 1:

| | |
|---|---|
| ATGAAACTCAGCGGTGTGGAACTGCGCCGGGTGCAGATGCCGCTCGTCGCCCCGTTCCGG | 60 |
| M K L S G V E L R R V Q M P L V A P F R | 20 |
| ACTTCGTTCGGCACCCAGTCGGTCCGCGAGCTCTTGCTGCTGCGCGCGGTCACGCCGGCC | 120 |
| T S F G T Q S V R E L L L L R A V T P A | 40 |
| GGCGAGGGCTGGGGCGAATGCGTGACGATGGCCGGTCCGCTGTACTCGTCGGAGTACAAC | 180 |
| G E G W G E C V T M A G P L Y S S E Y N | 60 |
| GACGGCGCGGAACACGTGCTGCGGCACTACTTGATCCCGGCGCTGCTGGCCGCGGAAGAC | 240 |
| D G A E H V L R H Y L I P A L L A A E D | 80 |
| ATCACCGCGGCGAAGGTGACGCCGCTGCTGGCCAAGTTCAAGGGCCACCGGATGGCCAAG | 300 |
| I T A A K V T P L L A K F K G H R M A K | 100 |
| GGCGCGCTGGAGATGGCCGTGCTCGACGCCGAACTCCGCGCGCACGAGAGGTCGTTCGCC | 360 |
| G A L E M A V L D A H L R A H E R S F A | 120 |
| GCCGAACTCGGATCGGTGCGCGATTCTGTGCCGTGCGGCGTTTCGGTCGGGATCATGGAC | 420 |
| A K L G S V R D S V P C G V S V G I M D | 140 |
| ACCATCCCGCAACTGCTCGACGTCGTGGGCGGATACCTCGACGAGGGTTACGTGCGGATC | 480 |
| T I P Q L L D V V G G Y L D E G Y V R I | 160 |
| AAGCTGAAGATCGAACCCGGCTGGGACGTCGAGCCGGTGCGCGCGGTCCGCGAGCGCTTC | 540 |
| K L K I E P G W D V E P V R A V R E R F | 180 |
| GGCGACGACGTGCTGCTGCAGGTCGACGCGAACACCGCCTACACCCTCGGCGACGCGCCG | 600 |
| G D D V L L Q V D A M T A Y T L G D A P | 200 |
| CAGCTGGCCCGGCTCGACCCGTTCGGCCTGCTGCTGATCGAGCAGCCGCTGGAAGAGGAG | 660 |
| Q L A R L D P F G L L L I E Q P L E E E | 220 |
| GACGTGCTCGGCCACGCCGAACTGGCCCGCCGGATCCAGACACCGATCTGCCTCGACGAG | 720 |
| D V L G H A E L A R R I Q T P I C L D E | 240 |
| TCGATCGTGTCGGCGCGCGCGGCGGCGGACGCCATCAAGCTGGGCGCGGTCCAAATCGTG | 780 |
| S I V S A R A A A D A I K L G A V Q I V | 260 |
| AACATCAAACCGGGCCGCGTCGGCGGGTACCTGGAAGCGCGGCGGGTGCACGACGTGTGC | 840 |
| N I K P G R V G G Y L E A R R V H D V C | 280 |
| GCGGCGCACGGGATCCCGGTGTGGTGCGGCGATATGATCGAGACCGGCCTCGGCCGGGCG | 900 |
| A A H G I P V W C G D M I E T G L G R A | 300 |
| GCGAACGTCGCGCTGGCCTCGCTGCCGAACTTCACCCTGCCCGGCGACACCTCGGCGTCG | 960 |
| A N V A L A S L P N F T L P G D T S A S | 320 |
| GACCGGTACTACAAAACCGACATCACCGAGCCGTTCGTGCTCTCCGGCGGCCACCTCCCG | 1020 |
| D R Y Y K T D I T E P F V L S G G H L P | 340 |
| GTGCCGACCGGACCGGGCCTCGGCGTGGCGCCGATTCCGGAGCTGCTGGACGAGGTGACC | 1080 |
| V P T G P G L G V A P I P E L L D E V T | 360 |
| ACGGCAAAGGTGTGGATCGGTTCGTAG | 1107 |
| T A K V W I G S * | 369 |

The two mutations, G291D and F323Y have re-sculpted the acyl binding pocket, previously evolved by nature for bonding of a succinyl side group. These changes have increased the acyl racemase activity to a higher level than that of the wild type. The increased racemase activity of the mutated enzyme has been shown to be approximately five times than that of the wild type.

U.S. Patent Application Publication No. 2003/0059816 A1 relates to methods for identifying enzymes with N-acyl amino acid recemase activity from microbial gene libraries. That publication also relates to methods of creating new racemases by directed evolution from related enzyme activities. Although the publication discloses mutated NAAARs, the mutation is not specific. The NAAARs are produced by random mutagenesis. Also the publication does not exemplify the activity of the NAAAR in a dynamic kinetic resolution method of producing enantiomerically pure amino acid from its N-acyl derivative, but is more related to randomly mutating enzymes with racemase activity and a method for selecting the most active racemase.

An aspect of the present application provides a synthesis of enantiomerically pure amino acid from its N-acyl amino acid derivative, via a dynamic kinetic resolution method. In the reaction, racemic N-acyl amino acid is treated with an acylase enzyme in the presence of G291D F323 Y Amycolatopsis sp. TS-1-60 NAAAR to afford enantiomerically pure amino acid in good yield. The overall reaction is shown as Scheme 3

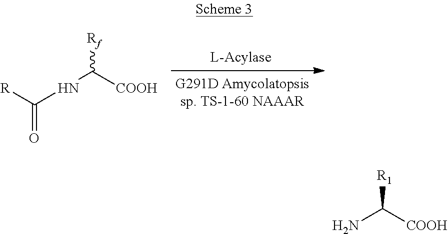

wherein,

R₁=alpha-radical of a natural or synthetic amino acid

### R=C₁-C₄

For example, in embodiments N-acetyl-DL-methionine is reacted with L-acylase and G291D F323Y Amycolatopsis sp. TS-1-60 NAAAR, in the presence of 10 mM Tris:HCl (pH 8.0) and 5 mM CoCl₂, at about 60° C. After completion of the reaction, 85% of L-methionine can be isolated from the reaction mixture (see Example 7, Table 2). Hence, G291D F323Y Amycolatopsis sp. TS-1-60 NAAAR can be successfully used in an industrial process for the production of enantiomerically pure amino acids from their racemic N-acyl derivatives, with an increased yield.

An aspect of the present application relates to a mutated Amycolatopsis sp. TS-1-60 NAAAR that shows improved activity of the enzyme compared with the wild type Amycolatopsis sp. TS-1-60 NAAAR.

Table 1 in Example 7 shows the specific activity of different NAAARs (namely, wild type, G291E mutated, G291D mutated, G291D P200S F323Y mutated and G291D F323Y mutated) with respect to the two substrates N-acetyl-D-methionine and N-acetyl-L-methionine. It may be observed that the highest activity is achieved by the G291D F323Y mutated enzyme.

Table 1 also demonstrates that when the same amount of the wild type and the G291D F323Y mutated NAAAR enzymes are reacted with N-acetyl-D-methionine for the same time period, the wild type enzyme shows an activity of 21.07 moles, whereas the mutated enzyme shows an activity of 99.80 moles (a factor of 4.74 times greater). Similarly, for N-acetyl-L-methionine, the wild type shows an activity of 29.99 moles, whereas the mutated enzyme shows an activity of 143.11 (a factor of 4.77 times greater). These results show that G291D F323Y mutated enzyme is more active than that of the wild type. This significant increase in activity is very surprising since the wild type enzyme has been mutated at only two positions, namely G291 and F323.

As stated above, the previously reported NAAARs have very low activity compared to the activity of acylase enzyme. Hence, they are not practically useful for the industrial production of enantiomerically pure amino acids from N-acyl amino acids, via a dynamic kinetic resolution method. The increase in specific activity of G291D F323Y mutated NAAAR makes it possible to overcome problems of the prior processes. Thus, the G291D F323Y mutated NAAAR can be successfully used for the industrial production of enantiomerically pure amino acids from their N-acyl derivatives.

The increased activity of the mutated G291D F323Y NAAAR has led to about 60% increases in the production of L-methionine, from a reaction comprising N-acetyl methionine, mutated NAAAR and L-acylase via the dynamic kinetic resolution method, compared to a conventional kinetic resolution method of production of L-methionine from a reaction comprising N-acetyl methionine and acylase (see Table 2). In an experiment, when N-acetyl-DL-methionine is reacted with L-acylase, only 52% of L-methionine is obtained. But the addition of G291D F323Y mutated NAAAR to the reaction of N-acetyl-DL-methionine and L-acylase increases the yield of L-methionine to about 85%.

Table 2 also shows that the G291D F323Y mutated NAAAR not only improves the yield of L-methionine from N-acetyl-DL-methionine but also increases yields of L-alanine, L-leucine, and L-phenylalanine from their corresponding N-acetyl derivatives. It clearly points out that the mutated G291D F323Y NAAAR has wide range of substrate specificity. So the mutated G291D F323Y enzyme is not only industrially useful for producing L-methionine but also a number of other amino acids from the N-acetyl derivatives.

It is observed that the racemase activity of the mutated NAAAR is more than that of Rac101, which is a commercially available racemase enzyme. Table 2 shows that when N-acetyl-DL-leucine is reacted with L-acylase and Rac101, a 53% yield of L-leucine is obtained. In a similar reaction, when Rac101 is substituted with G291D F323Y NAAAR, the yield improves to 67%. In case of L-methionine, the increase in yield is much more significant. It shows a yield increase of more than 60% for G291D F323Y NAAAR, over that of Rac101.

The G291D F323Y Amycolatopsis sp. TS-1-60 NAAAR has a wide range of substrate specificity. It is useful for racemizing a wide range of N-acyl amino acid derivatives. The amino acids may be natural or synthetic. Some of the examples of amino acid substrates include, but are not limited to, N-acyl-D-methionine, N-acyl-L-methionine, N-acyl-D-alanine, N-acyl-L-alanine, N-acyl-D-leucine, N-acyl-L-leucine, N-acyl-D-phenyalanine, N-acyl-L-phenyalanine, N-acyl-D-isoleucine, N-acyl-L-isoleueine, N-acyl-D-valine, N-acyl-D-tryptophan, N-acyl-L-tryptophan, N-acyl-D-aspartic acid, N-acyl-L-aspartic acid, N-acyl-D-phenylglycine, N-acyl-L-phenylglycine, N-acyl-D-(4-fluorophenyl)glycine, N-acyl-L-(4-fluorophenyl)glycine, N-acyl-D-2-aminobutyrate, N-acyl-L-2-aminobutyrate, N-acyl-D-allylglycine, N-acyl-L-allylglycine. The acyl group can be any group comprising one to four carbon atoms. In specific embodiments, the acyl group is an acetyl group (—COCH₃).

Table 3 shows the efficiency of the G291D F323Y mutated NAAAR against a wide range of amino acids. All the reactions were performed at 60° C., 300 mM substrate, 100 mM Tris:HCL (PH 8.0), 5 mM CoCl₂. Minimum concentration of G291D F323Y mutated NAAAR was added and the reaction mass was analyzed after 8 hours. In case of N-acetyl D-methionine or N-acetyl-L-methionine, the reaction mass shows an enantiomeric excess of only less than about 4% after 8 hours, indicating that more than about 96% of the substrate was racemised. This proves the efficiency of G291D F323Y mutated NAAAR. Similarly, Table 3 shows the effectiveness of G291D F323Y imitated NAAAR for other substrates like N-acetyl-D-phenyalanine, N-acetyl-L-phenyalanine, N-acetyl-D-phenylglycine, N-acetyl-L-phenylglycine, N-acetyl-D-2-aminobutyrate, N-acetyl-L-2-aminobutyrate, N-acetyl-D-(4-fluorophenylglycine) and N-acetyl-D-allylglycine.

These results show that G291D F323Y Amycolatopsis sp. TS-1-60 NAAAR is useful for the production of enantiomerically pure amino acids from their N-acyl derivatives via a dynamic kinetic resolution method.

It has been observed that the mutated G291D F323Y Amycolatopsis sp. TS-1-60 NAAAR is active at about 20° C. to about 80° C., specifically at about 30° C. to about 70° C., and more specifically at about 37° C. to about 60° C.

The prior NAAARs are primarily of the wild types and they are reported to be inhibited by the substrate. As a result, yields of enantiomerically pure amino acids are lower with the wild type NAAARs. It has been found that the reported substrate inhibition of the wild type enzyme was due to lack of control of pH. It is surprisingly observed that the mutated G291D F323Y Amycolatopsis sp. TS-1-60 NAAAR shows highest activity at slightly basic pH values. The mutated G291D F323Y Amycolatopsis sp. TS-1-60 NAAAR shows high activity at pH 7.5-9, or at pH 7.5-8.5, or at pH 8. It is also observed that mutated G291D F323Y Amycolatopsis sp. TS-1-60 NAAAR does not inhibit up to about 300 mM of the substrate at pH 8. Therefore, the mutated G291D F323Y Amycolatopsis sp. TS-1-60 NAAAR can be used for the production of enantiomerically pure amino acids from racemic N-acyl amino acid via dynamic kinetic resolution methods, under industrially acceptable conditions.

A further aspect of the present application relates to the stereoinversion of a ‘low-cost’ (or unwanted) enantiomer of an amino acid or amino acid derivative into a ‘high-value’ (or desired) amino acid or amino acid derivative, such as is depicted in Scheme 4. This aspect will be particularly useful for converting a readily available natural L-amino acid into the less available unnatural D-amino acid, for example, converting L-serine to D-serine.

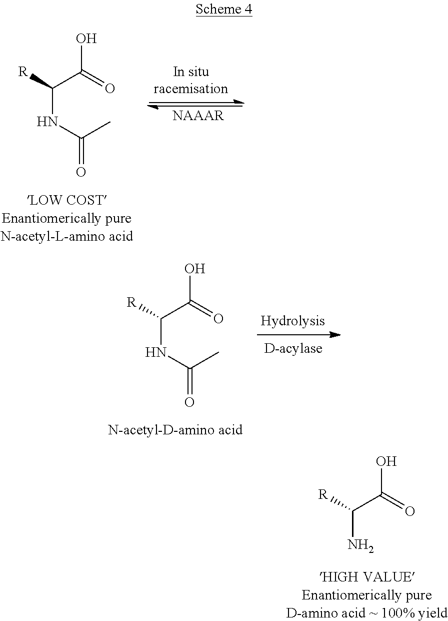

In general, N-acetyl derivatives of L-amino acids (enantiomerically pure or enantiomerically enriched) can be conveniently obtained from the L-amino acids by their reaction with acetic anhydride in the presence of base. The obtained compound can be subjected NAAAR/D-acylase coupled hydrolysis, which will provide a D-enantiomer of the starting amino acid. As a result the process can be regarded as formal stereoinversion of the starting amino acid.

Certain specific aspects and embodiments will be further explained in the following examples, which are being provided only for the purpose of illustration, and the scope of this application is not limited thereto.

### Example 1

### PCR Based Point Mutation at G291

Saturation mutagenesis was carried out at position G291 of the wild type (WT) Amycolatopsis NAAAR gene. Mutagenesis was carried out using a mutagenic forward primer encoding a degenerate NNK codon instead of the WT GGG and a non-mutagenic reverse primer encoding the end of the NAAAR gene. The ˜200 bp PCR product was used as a mega primer and pTTQ18 WT NAAAR used as the vector template in a mega primer based mutagenesis PCR. The resulting PCR product was digested at 37° C. with Dpn 1 to remove template DNA for 4 hours. Plasmids were screened using the SET21 bacterial strain. Screening was performed at 37° C. on Davis minimal agar plates supplemented with 1 mM N-acetyl-D-methionine, 0.4% glucose, 100 μM CaCl₂, 0.25 mM IPTG, and 30 μg/mL chloramphenicol. After electro-competent transformation, cells were washed three times with H₂O to remove all rich media from cell mixtures. 100% of the cell mixture was spread split between four agar plates. Plates were incubated overnight and then colony size was judged visually. The largest colony from each plate was re-streaked onto replica 1 mM N-acetyl-D-methionine plates to confirm enhanced growth, compared to WT-3 colonies. These were sequenced and all found to contain the aspartic acid, GAC codon at position 291.

### Example 2

### PCR Based Point Mutation at F323

The F323Y mutation was discovered with error prone PCR using pET20b NAAAR G291D as the template. The NAAAR G291D gene was amplified using a commercial error prone PCR kit (Genemorph II, Startagene) with a mutagenic rate corresponding to 1 amino acid mutation per gene. The initial mutagenic PCR product was cloned into pET20b using a mega primer based PCR with pET20b NAAAR (WT) as the template. Screening was performed in a DE3 lysigenic strain of SET21. Colonies were selected on Davies minimal agar plates supplemented with 500 μM N-acetyl-D-methionine, 100 μg/mL ampicillin, and 30 μg/mL chloramphenicol. Cells were washed three times with H₂O before spreading on plates to remove all rich media from cell mixtures. The largest colonies from each plate were re-streaked onto replica 500 μM N-acetyl-D-methionine plates to confirm enhanced growth compared to NAAAR G291D. Two larger growing colonies were found to contain the tyrosine, TAC codon at position 323.

### Example 3

### Purification of Wild Type and Mutated NAAARs

WT, G291D and G291D F323D NAAAR were purified by the same method. The corresponding pET 20b plasmid was transformed into BL21 (DE3) and a single colony from this was used to inoculate 500 mL LB (100 μg/mL ampicillin). This culture was grown for 24 hours at 37° C. with no induction. Cells were then collected via centrifugation (15 minutes, 4000 g) and lysed immediately with 10 minutes of sonication (30 seconds on, then 30 seconds off) in 50 mM tris:HCl (pH 8.0), 100 mM NaCl, Roche complete EDTA free protease inhibitor tablet, and 2 mg/mL lysozyme. This was then clarified with centrifugation (1 hour, 12000 G, 4° C.) and the supernatant was filtered through a 0.45 μm filter. The filtered supernatant was then loaded onto a HiPrep 16/10 FF Q anion exchange column attached to an AKTA system. This column was equilibrated with 50 mM Tris:HCl (pH 8.0), 100 M NaCl. Proteins were then eluted with the following gradient with 50 mM Tris:HCl (pH 8.0), 100 M NaCl: 0 to 25% over 1 column volume, 25 to 45% over 8 column volumes, and 45 to 100% over 1 column volume. Fractions containing NAAAR (judged by SDS PAGE gel) were pooled and concentrated to ˜1 mL before being loaded onto a Sephadex 300 size exclusion column equilibrated with 50 mM Tris:HCl (pH 8.0) 100 mM NaCl. Fractions thought to be containing NAAAR were pooled and protein concentration determined via Abs280. Protein was stored at 4° C. before being assayed.

### Example 4

### Measurement of Specific Activity of NAAAR

Measurement of specific activity was made by assaying purified WT, G291D and G291D F323Y enzymes. Assays were performed in 100 mM Tris:HCl (pH 8.0), 5 mM CoCl₂ with to 300 mM N-acetyl-methionine. The substrate were prepared in 100 mM Tris:HCl (pH 8.0) and the pH adjusted again after addition of substrate, this was found to be beneficial for optimizing activity above 30 mM. The final 100 mM Tris:HCl in the reaction buffer was made up with 50 mM coming from the substrate solution. Enzyme and buffer were incubated at 60° C. for 5 minutes before addition of NAAAR to the reaction. The reaction was left at 60° C. for 3 minutes before being terminated by addition of 50 μL of reaction into 950 μL 0.05 M HCl. This was then boiled for 5 minutes to precipitate all protein and the solution clarified with centrifugation (3 minutes, 11000 G). The supernatant was 0.45 μm filtered before analysis with chiral HPLC. HPLC was carried out on an Agilent 1100 system using a Chirobiotic T column at 40° C. The gradient was an isocratic mobile phase of 75% 0.01% TEAA and 25% methanol. Peaks were monitored at 210 nm. Injection volume was 5 μL. Analysis were carried out using Chemstation software.

### Example 5

### Purification of L-Acylase

L-acylase was purified by expression in BL21 (DE3) cells grown for 24 hours in auto-induction media (100 μg/mL Ampicillin). Cells were then collected via centrifugation (15 minutes, 4000 g) and lysed immediately with 10 minutes of sonication (30 seconds on, then 30 seconds off) in 50 mM Tris:HCl (pH 8.0), Roche complete EDTA free protease inhibitor tablet, and 2 mg/mL lysozyme. This solution was incubated at 60° C. for 60 minutes. This was then clarified with centrifugation (1 hour, 12000 G, 4° C.) and the supernatant was filtered through a 0.45 μm filter. The filtered supernatant was then loaded onto a HiPrep 16/10 FF Q anion exchange column attached to an AKTA system. The column was equilibrated with 50 mM Tris:HCl (pH 8.0). Proteins were then eluted with the following gradient with 50 mM (pH 8.0), 1M NaCl: 0-25% over 1 column volume, 25-45% over 8 column volumes, and 45-to 100% over 1 column volume. Fractions containing L-acylase were judged by SDS-PAGE analysis.

### Example 6

### Biotransformation

Small scale biotransformations were carried out to test NAAAR compatibility with both the L-acylase and other amino acids. B:A21 cells were transformed with pET20b NAAAR G291D F323Y and a plasmid encoding an L-acylase (ampicillin resistant). A single NAAAR colony was used to inoculate 5 mL of LB (100 μg/mL ampicillin) and a single L-acylase colony used to inoculate 5 mL of auto-induction media (10 g/L peptone, 5 g/L yeast extract, 50 mM (NH₄)₂SO₄, 100 mM KH₂PO₄, 100 mM of Na₂HPO₄, 0.5% glycerol, 0.05% glucose, 0.2% lactose, 1 mM MgSO₄, 100 μg/mL ampicillin). Both cultures were grown at 37° C. for 24 hours before 1 mL of each was removed and added to 8 mL of biotransformation reaction buffer (final concentration: 100 mM Tris:HCl (pH 8.0), 5 mM CoCl₂ and 300 mM substrate). The pH of reaction buffer after addition of substrate was readjusted to 8.0. The biotransformation was incubated at 60° C. for several hours with 1 mL samples removed at specific time points to monitor the reaction progress. These samples were clarified with centrifugation (2 minutes, 11000 G) and 50 μL supernatant added to 950 μL 0.05 M Samples were then prepared and analysed via chiral HPLC as explained in Example 4.

### Example 7

### Comparison of Activity of NAAAR G291 D F323Y with Rac101

To compare the activity of commercially available Rac101 with G291D F323Y NAAAR, purified enzymes were used in place of cells. 0.1 mg of each enzyme was included in the 1 mL reaction. The condition, preparation, and analysis of samples were similar to those of Example 5.

The results of the experiments are shown in Tables 1 and 2. Table 1 compares the specific activity of different NAAARs and Table 2 shows the yields from NAAAR and Rae 101 coupled biotransformations using different substrates.

TABLE 1
Activity of variant NAAARs with N-acetyl methionine

Specific Activity (μmoles/minute/mg)

| Enzyme | N-acetyl-D-methionine | N-acetyl-L-methionine |
|---|---|---|
| WT | 21.07 | 29.99 |
| G291E | 40.14 | 71.44 |
| G291D | 93.87 | 118.8 |
| G291D | 49.82 | 58.96 |
| P2000S | | |
| F323Y | | |
| G291D | 99.8 | 143.11 |
| F323Y | | |

TABLE 2
Yield from NAAAR and Rac101 coupled biotransformations

| | | % Conversion to L-amino acid^{(a), (b)} | | |
|---|---|---|---|---|
| | | | | NAAAR |
| | | | | G291D |
| | Concentration | | RAC101 + | F323Y + |
| Substrate | (g/L) | L-acylase | L-acylase | L-acylase |
| N-acetyl-DL- | 48 | 52 | 52 | 85 |
| methionine | | | | |
| N-acetyl-DL- | 33 | 42 | 42 | 65 |
| alanine | | | | |
| N-acetyl-DL- | 44 | 50 | 53 | 67 |
| leucine | | | | |
| N-acetyl-DL- | 52 | 45 | 44 | 57 |
| phenylalanine | | | | |

^{(a)}No D-amino acid was detected in any biotransformation.
^{(b)} Reaction conditions: 250 mM substrate, 100 mM Tris: HCl (pH 8.0), 5 mM CoCl₂, 60° C.

### Example 8

### Racemization of Wide Variety of N-Acyl Amino Acids by G291D F323Y NAAAR

All reactions were carried out at 60° C., 300 mM substrate, 100 mM Tris:HCl (pH 8.0), 5 mM CoCl₂. G291D F323Y NAAAR was added at minimum concentration and the reactions were carried out for 8 hours. After 8 hours the enantiomeric excess (% ee) of the reaction mass was analyzed. The results of the experiments are shown in Table

TABLE 3
Racemization of wide variety of N-
acyl amino acids by G291D F323Y NAAAR

| | Substrate | G291D F323Y | Enantiomeric |
|---|---|---|---|
| | Conc. | NAAAR Conc. | excess of reaction |
| Substrate | (g L⁻¹) | (kU L⁻¹) | mass (% ee) |
| N-acetyl-D- | 50 | 60 | <4 |
| methionine | | | |
| N-acetyl-L- | 50 | 60 | <4 |
| methionine | | | |
| N-acetyl-L- | 60 | 60 | <4 |
| phenylglycine | | | |
| N-acetyl-D- | 60 | 60 | <2 |
| phenylglycine | | | |
| N-acetyl-D-(4- | 60 | 60 | <3 |
| fluorophenylglycine) | | | |
| N-acetyl-D- | 60 | 200 | <2 |
| phenylalanine | | | |
| N-acetyl-L- | 60 | 200 | <14 |
| phenylalanine | | | |
| N-acetyl-D-2- | 44 | 200 | <23 |
| aminobutyrate | | | |
| N-acetyl-L-2- | 44 | 200 | <19 |
| aminobutyrate | | | |
| N-acetyl-D- | 50 | 30 | <0.1 |
| allylglycine | | | |

### Example 9

### Stereoinversion of L-Serine into D-Serine and the Formation of N-Boc-D-Serine Derivative in a One-Pot Process

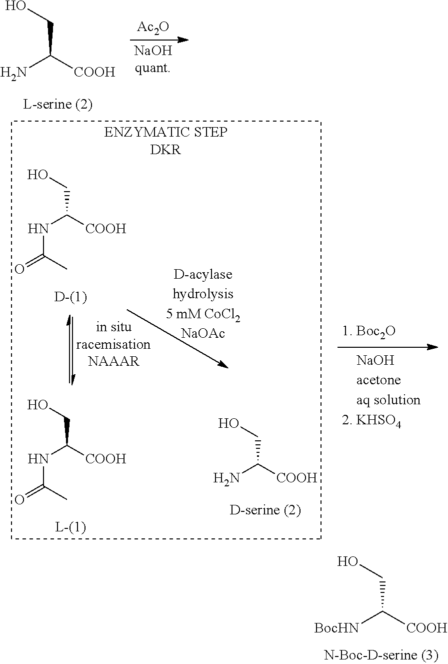

L-serine (142.7 mmol, 15.0 g) was dissolved in a chilled solution of NaOH (14.4 g, 360 mmol) in water (25 mL) and cooled. Acetic anhydride (15 mL) was added dropwise, maintaining the temperature below 25° C., and then the mixture was stirred for 1 hour. 100 mL of EtOH was added and the slurry was stirred for 1 hour at ambient temperature to decompose unreacted Ac₂O. The white precipitate (Na, 15 g) was removed by filtration. The filtrate was evaporated in vacuo to give ˜56 g of crude product. The residue was redissolved in 120 mL of MeOH and 80 mL of EtOH and acidified to pH 4.5 with cone. HCl upon cooling in an ice bath. A white precipitate (NaCl, 25 g) was removed by filtration. 100 mL of toluene was added to the filtrate and the residue was evaporated to give the product in a 1:1.2 molar ratio with H (by NMR). The residue was dissolved in 500 mL of H₂O and 609 mg of CoCl₂ and 300 mg of MgSO₄ were added. The mixture was warmed to 40° C. and the pH was adjusted to 8.0. 30 mL of NAAAR G291D F323Y cell free extract in 10 mM NaOAc (1500 U) was added. The racemisation reaction was monitored by chiral HPLC. After 3 hours the conversion reached 50% and 250 μL of Alcaligenes sp. D-aminoacylase was added (360 U) and the mixture was stirred at 40° C. overnight, with pH maintained at 7.8-8 using 2 M NaOH. After 18 hours from the addition of the acylase the conversion reached ˜80%. Another 25 μL of Alcaligenes sp. D-aminoacylase (36 U) was added. After stirring for another 3 hours the reaction reached ˜90% conversion. The mixture was cooled to 5° C. and the pH was adjusted to 2.3 using 3M HCl and it was then centrifuged (30 minutes, 8000 rpm) to remove the enzyme. The aqueous phase was washed with 2×100 mL Et. The organic phase was then washed with 100 mL of 1M HCl. Combined aqueous layers were cooled to 5° C. and adjusted to pH 12 using 46% NaOH. Boc₂O (38.6 g, 1.2 eq) dissolved in 100 mL of acetone was added dropwise to the cooled reaction mixture, keeping the temperature <10° C. The mixture was stirred overnight at ambient temperature. The aqueous layer was cooled to 5° C. and acidified to pH 3 using 1M KHSO₄ (gas evolved). It was extracted with 1500 mL of Et. The organic layer was washed with brine, dried over MgSO₄ and evaporated to dryness to give a crude product (˜33 g, 94% ee). It was recrystallized from MTBE/heptane, upon addition of 5 mg of commercial N-Boc-D-serine to aid the crystallization. 16.42 g of pure product was obtained as white crystals (96% ee, 56% yield for 3 steps from serine).

### Example 10

### Stereoinversion of N-Acetyl-D-Allylglycine into L-Allylglycine

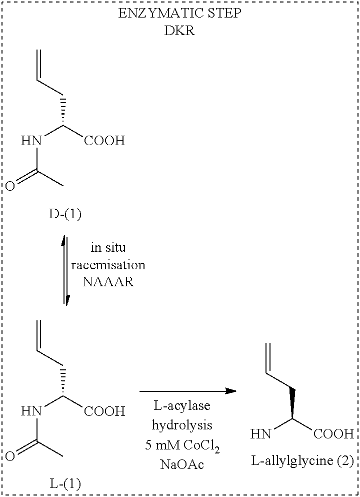

N-Acetyl-D-allylglycine (50.0 g, 318 mmol) was dissolved in 400 mL of H₂O. The solution was warmed up to 60° C. and the pH was adjusted to 8.0 using 46% NaOH. 236 mg of CoCl₂ and 136 mg of ZnCl₂ were added. 10 mL of NAAAR G291D F323Y cell free extract in 10 mM NaOAc (500 U) was added and the racemisation reaction was monitored by chiral HPLC. After 1 hour the conversion reached 30% and 750 mg of Thermocaccus litoralis L-aminoacylase (30 kU) in water containing another 236 mg of CoCl₂ and 136 mg of ZnCl₂ was added. The mixture was stirred at 60° C. and the pH was maintained at 8-8.2 using 5 M NaOH. After 3.5 hours and 5.5 hours another 10 mL of NAAAR G291D F323Y cell free extract in 10 mM NaOAc (500 U) were added (30 mL in total, 1500 U). The mixture was stirred overnight to reach 80% conversion of the substrate into L-allylgycine (>95% ee).

We claims:

1. N-acyl amino acid racemase (NAAAR) comprising an amino acid sequence that is at least 90% identical to SEQ ID No. 1.

2. The NAAAR of claim 1, wherein NAAAR is mutated Amycolatopsis sp. TS-1-60 NAAAR of SEQ. ID No. 1.

3. A process for the preparation of enantiomerically pure amino acids comprising treating acyl derivative of an amino acid with NAAAR having an amino acid sequence that is at least 90% identical to SEQ ID No. 1 and an acylase enzyme.

4. The process of claim 3, wherein the reaction is performed at about 20° C. to about 80° C.

5. The process of claim 4, wherein the reaction is performed at about 30° C. to about 70° C.

6. The process of claim 3, wherein the reaction is performed at a pH of about 7.5 to about 9.

7. The process of claim 6, wherein the reaction is performed at a pH of about 8.

8. The process of claim 3, wherein acyl derivative of amino acid comprises one to four carbon atoms in the acyl group.

9. The process of claim 8, wherein the acyl group is an acetyl group.

10. The process of claim 3, wherein the substrate concentration is about 300 mM.

11. The process of claim 3, wherein the substrate concentration is at least about 50 mM.

12. The process of claim 3, wherein amino acid is selected from a group of D-methionine, L-methionine, D-alanine, L-alanine, D-leucine, L-leucine, D-phenyalanine, L-phenylalanine, D-isoleucine, L-isoleucine, D-valine, L-valine, D-tryptophan, L-tryptophan, D-aspartic acid, L-aspartic acid, D-phenylglycine, L-phenylglycine, D-(4-fluorophenyl)glycine, L-(4-fluorophenyl)glycine, D-2-aminobutyrate, L-2-aminobutyrate, D-allylglycine, L-allylglycine, L-serine and D-serine.

13. The use of NAAAR having an amino acid sequence that is at least 90% identical to SEQ ID No. 1 for the production of enantiomerically pure amino acids.

14. The use of NAAAR having an amino acid sequence that is at least 90% identical to SEQ ID No. 1 for the stereoinversion of an amino acid.

15. The use of NAAAR having an amino acid sequence that is at least 90% identical to SEQ ID No. 1 for the production of enantiomerically pure amino acids from a racemic mixture of amino acid.


SEQUENCE LISTING (1 sequence)

SEQ ID NO: 1 - protein, 368 aa, amycolatopsis sp Ts 1- 60
MKLSGVELRRVQMPLVAPFRTSFGTQSVRELLLLRAVTPAGEGWGECVTMAGPLYSSEYNDGAEHVLRHYLIPALLAAEDITAAKVTPLLAKFKGHRMAKGALEMAVLDAELRAHERSFAAELGSVRDSVPCGVSVGIMDTIPQLLDVVGGYLDEGYVRIKLKIEPGWDVEPVRAVRERFGDDVLLQVDANTAYTLGDAPQLARLDPFGLLLIEQPLEEEDVLGHAELARRIQTPICLDESIVSARAAADAIKLGAVQIVNIKPGRVGGYLEARRVHDVCAAHGIPVWCGDMIETGLGRAANVALASLPNFTLPGDTSASDRYYKTDITEPFVLSGGHLPVPTGPGLGVAPIPELLDEVTTAKVWIGS